(19) United States
(12) Patent Application Publication
(10) Pub. No.: US 2007/0238942 A1
(43) Pub. Date: Oct. 11, 2007

(54) APPARATUS AND METHOD FOR DETECTING GASTROESOPHAGEAL REFLUX DISEASE (GERD)

(75) Inventor: Richard A. Baylor, Champaign, IL (US)

Correspondence Address: BELL, BOYD & LLOYD, LLP, P.O. Box 1135, CHICAGO, IL, 60690, US

(73) Assignee: ESOPHAMET CORP., Champaign, IL (US)

(21) Appl. No.: 11/459,454

(22) Filed: Jul. 24, 2006

Related U.S. Application Data
(60) Provisional application No. 60/790,701, filed on Apr. 10, 2006.

(51) Int. Cl. A61B 5/00

(52) U.S. Cl. 600/309

(57) ABSTRACT

A method and an apparatus are disclosed for detecting gastroesophageal reflux disease (GERD) in the esophagus of a patient. A capsule with pH-sensitive dissolving properties is placed in the lower portion of the esophagus of the patient. The capsule is removed after a period of time, such as about 24 hours. The change in weight or volume of the capsule, or both, allows detection of acid reflux, i.e., the presence of acid in the esophagus for even a relatively short duration during the period of time. If the weight or volume change is significant, the capsule allows diagnosis of GERD. Alternatively, an amount of time during which the capsule was exposed to acid in the esophagus may be determined by dissolving the capsule remaining and using a solution property, such as conductivity, pH, color, or turbidity.

This application claims the benefit of the filing date under 35 U.S.C. 119(e) of Provisional Application 60/790,701, filed Apr. 10, 2006, which is hereby incorporated by reference in its entirety.

### FIELD OF THE INVENTION

The field of the invention is that of detecting and diagnosing a disease.

### BACKGROUND

Gastroesophageal reflux occurs when stomach acid intermittently surges into the esophagus. It is common for most people to experience this acid reflux occasionally as heart bum. Gastroesophageal reflux disease (GERD) is a clinical condition in which the reflux of stomach acid into the esophagus is frequent enough and severe enough to impact a patient's normal functioning or to cause damage to the esophagus. GERD is sometimes also referred to as “reflux” or “reflux esophagitis.”

It has been estimated by the U.S. Department of Health and Human Services that about seven million people in the United States suffer from GERD. The incidence of GERD increases after the age of 40, and more than 50 percent of those afflicted with GERD are between the ages of 45-64. (Statistics from Digestive Diseases in the United States: Epidemiology and Impact, National Digestive Diseases Data Working Group, James E. Everhart, MD, MPH, Editor, U.S. Department of Health and Human Services, Public Health Service, National Institutes of Health, NIH Publication No. 94-1447, May 1994.) For general information about GERD see the following: Fennerty, M. B., Sampliner, R. E., Gastroesophageal reflux disease, Hospital Medicine, 29(4): 28-40 (1993); and Orlando, R. C., Reflux esophagitis, in Textbook of Gastroenterology, 1: 1123-1147, Yamada, T., ed., J. B. Lippincott Co., Philadelphia, Pa. (1991).

In the lower part of esophagus, where the esophagus meets the stomach, there is a muscular valve called the lower esophageal sphincter (LES). Normally, the LES relaxes to allow food to enter into the stomach from the esophagus. The LES then contracts to prevent stomach acids from entering the esophagus. In GERD, the LES relaxes too frequently or at inappropriate times allowing stomach acids to reflux into the esophagus.

The most common symptom of GERD is heartburn. Acid reflux also leads to esophageal inflammation, which causes symptoms such as odynophagia, or painful swallowing, and dysphagia, or difficulty swallowing. Pulmonary symptoms such as coughing, wheezing, asthma, or inflammation of the vocal cords or throat may occur in some patients. More serious complications from GERD include esophageal ulcers and esophageal stricture, or narrowing of the esophagus. The most serious complication from chronic GERD is a condition called Barrett's esophagus in which the epithelium of the esophagus is replaced with abnormal tissue. Barrett's esophagus is a risk factor for the development of cancer of the esophagus.

Accurate diagnosis of GERD is difficult but important. Accurate diagnosis allows identification of individuals at high risk for developing the complications associated with GERD. It is also important to be able to differentiate between gastroesophageal reflux, other gastrointestinal conditions, and various cardiac conditions. For example, the similarity between the symptoms of a heart attack and heart burn often lead to confusion about the cause of the symptoms.

Several methods are currently being used to diagnose GERD and its associated complications. In healthy subjects, esophageal pH values are greater than pH 4 most of the time, and are lower than pH 4 only a very small percentage of the time. Therefore, an esophageal pH of less than pH 4 is generally used as the threshold to determine the presence of excessive acid reflux. See, e.g. H. G. Dammann, M.D., University of Hamburg, Hamburg Science Institute for Clinical Research “The Relevance of Acidity Measurements in the Management of Gastro-oesophageal Reflux Disease,” Research & Clinical Forums, 20(2):19-26 (1998) (healthy subjects had an esophageal pH of less than pH 4 approximately 1.5% of the time within a twenty-four hour period); and Jamieson J. R., Stein H. J., DeMeester T. R., Bonavina L., Schwizer W., Hinder R. A., and Albertucci M., “Ambulatory 24-hour esophageal pH monitoring: normal values, optimal thresholds, specificity, sensitivity, and reproducibility,” Am. J. Gastroenterol., 87(9):1102-11 (1992).

It is difficult to accurately test esophageal pH because the episodes of acid reflux into the esophagus are sporadic even in patients with severe reflux disease. Within a twenty-four hour period, the episodes of reflux may only occur about 10 to 15 percent of the time. See, e.g. Fink, S. M., and McCallum, R. W., “The role of prolonged esophageal pH monitoring in the diagnosis of gastroesophageal reflux,” JAMA, 252(9):1160-64 (1984) (during 24-hour pH monitoring, the mean percentage time that pH was less than pH 4.0 was approximately 13.2% for GERD patients, and approximately 2.9% for normal subjects); and Vitale, G. C. et al., “Computerized 24-hour ambulatory esophageal pH monitoring and esophagogastroduodenoscopy in the reflux patient: a comparative study,” Ann. Surg., 200(6):724-728 (1984) (the mean length of time of reflux below pH 4 was 5.41 minutes/hour, or approximately 9 percent of the time, in patients with reflux symptoms, and 0.70 minutes/hour, or approximately 1.2 percent of the time, in normal subjects). At any given time, the esophageal pH is likely to be normal. Therefore, it is important to assess the total time during which the esophagus is exposed to a clinically significant low pH over an extended period of time, such as twenty-four hours.

These studies indicate that in patients who exhibit symptoms of reflux, the percentage of time during which the esophageal pH is less than pH 4 may vary, and may be in the range of only ten to fifteen percent of the time. In normal subjects, esophageal pH is less than pH 4 only a very small percentage of the time, typically between one to three percent of the time. Therefore, an esophageal pH of less than about pH 4 for about five or more percent of the time is indicative of significant reflux. The percentage of time during which the patient's esophagus is exposed to pH levels less than about pH 4 is correlated with the severity of the disease—the greater the time of exposure, the more severe the condition. An esophageal pH of less than about pH 4 for about ten or fifteen percent of the time is indicative of more severe GERD.

Esophageal manometry, esophageal endoscopy, and esophageal pH monitoring are standard methods of measuring esophageal exposure to stomach acids and are currently used to diagnose GERD. Conventional pH monitoring involves placing a pH probe in the esophagus. Preferably, esophageal pH monitoring would take place over a twenty-four hour period.

Several methods of gastrointestinal pH monitoring have been used including intubation methods, ingestible capsules, glass electrodes, and radiotelemetry pills. Intubation involves the insertion of a tube into the patient. The tube is inserted through the nose and into the gastrointestinal tract of the patient. There may be a device at the inserted end of the tube which allows retrieval of a sample for further analysis, as disclosed in U.S. Pat. No. 4,735,214. Alternatively, the tube may be associated with an acid-base indicator, as disclosed in U.S. Pat. No. 3,373,735. Intubation methods are generally used for pH monitoring at a specific time, which does not allow for a determination of time exposure to clinically significant low pH. Intubation is also painful and uncomfortable, and it must be carried out in a hospital or clinical setting.

Tubeless methods and ingestible capsules have also been used to measure gastrointestinal pH. Ingestible capsules have been used to determine pH levels at a specific time and to retrieve samples from the gastrointestinal tract of a patient for further analysis. An ingestible capsule using an ion-exchange color indicator has also been suggested for use in twenty-four hour monitoring of esophageal pH, as disclosed in U.S. Pat. No. 4,632,119.

Electronic pH monitoring devices have also been used. A glass electrode or a radiotelemetry pill is introduced nasally or orally and is positioned in the esophagus proximal to the LES. The pH probe is connected to a microprocessing unit and pH levels are continuously recorded over a twenty-four hour period. (See, Colson, et al., “An Accurate, Long-Term, pH-Sensitive Radio Pill for Ingestion and Implantation,” Biotelemetry Patient Monitg., 8: 213-227 (1981). This type of monitoring may take place in a hospital or clinic.

Alternatively, with a portable microprocessor, the patient may be ambulatory. Even with a portable unit, the procedure is uncomfortable, and the apparatus is cumbersome. The probe is passed through the nose, and wires extending from the probe course over the face, across the chest, and attach to a recording device worn on a belt. Both the appearance of the apparatus and the discomfort it causes may restrict a patient from engaging in his normal daily activities, thus interfering with the diagnostic result. Additionally, electronic pH monitoring is expensive and requires computer analysis of the data gathered.

Another technique that has been disclosed in U.S. Pat. No. 6,475,145, using minute, safe amounts of radioactive material. However, the logistics of handling and managing even small, safe amounts of radioactive material, and subsequent detection is burdensome and expensive.

### BRIEF SUMMARY

One embodiment is a device for determining a duration of exposure of a patient's esophagus to pH levels clinically significant for gastroesophageal reflux disease. The device includes a capsule subject to pH-dependent degradation at pH of about pH 4, and is of a size to be readily swallowable, wherein the capsule comprises a reaction product of a cellulose, a polysaccharide, or a cationic polymer with an organic acid. The device also includes a cord, the cord having a proximal end and a distal end, the proximal end being connected to the capsule, whereby the cord allows positioning and retrieval of the capsule.

Another embodiment is a device for determining a duration of exposure of a patient's esophagus to pH levels clinically significant for gastroesophageal reflux disease. The device includes a capsule subject to pH-dependent degradation at pH of about pH 4, and of a size to be readily swallowable, the capsule further comprising a reaction product of an acid and a cationic polymer based on dimethylaminoethyl methacrylate and neutral methacrylic esters. The device also includes a cord, the cord having a proximal end and a distal end, the proximal end being connected to the capsule, whereby the cord allows positioning and retrieval of the capsule.

Another embodiment is a method of detecting gastroesophageal reflux disease. The method include steps of providing a gastroesophageal diagnostic device, said gastroesophageal diagnostic device comprising a capsule having a cord attached thereto, the capsule being subject to pH-dependent degradation at pH levels of about pH 4; measuring at least one of a mass and a volume of the capsule; introducing the gastroesophageal diagnostic device into an esophagus of a patient; positioning the gastroesophageal diagnostic device in the esophagus of the patient such that the capsule is positioned in the lower one-third of the esophagus; leaving the gastroesophageal diagnostic device in the esophagus of the patient for a time, whereby the capsule loses a portion of at least one of the mass and the volume in a pH-dependent manner; and removing the gastroesophageal diagnostic device from the patient after the time. The method also includes a step of determining an exposure time during which the capsule was exposed to pH levels of about pH 4, wherein the reduction in the amount of the mass or the volume correlates to the exposure time during which the capsule was exposed to pH levels of about pH 4, and wherein exposure of the capsule to pH levels of about pH 4 for at least a determined percent of the time the device was left in the esophagus is indicative of gastroesophageal reflux disease.

Another embodiment is a method of detecting gastroesophageal reflux disease. The method includes steps of: providing a gastroesophageal diagnostic device, said gastroesophageal diagnostic device comprising a capsule having a cord attached thereto, the capsule being subject to pH-dependent degradation at pH levels of about pH 4; introducing the gastroesophageal diagnostic device into an esophagus of a patient; positioning the gastroesophageal diagnostic device in the esophagus of the patient such that the capsule is positioned in the lower one-third of the esophagus; leaving the gastroesophageal diagnostic device in the esophagus of the patient for a time, whereby the capsule loses a portion of at least one of a mass and a volume of the device in a pH-dependent manner; removing the gastroesophageal diagnostic device from the patient after the time; and determining an exposure time during which the capsule was exposed to pH levels of about pH 4 by a method selected from the group consisting of color, turbidity, pH, and conductivity, wherein exposure of the capsule to pH levels of about pH 4 for a minimum time during the time the device is left in the esophagus is indicative of gastroesophageal reflux disease.

There are many embodiments of the present invention. The following description and figures are intended to be merely illustrative, rather than limiting, of the embodiments.

### BRIEF DESCRIPTION OF THE DRAWINGS

FIG. 1 is a schematic illustration of the gastrointestinal diagnostic capsule of the present invention illustrating the manner in which it is inserted into a patient;

FIG. 2 is a schematic illustration of the gastrointestinal diagnostic capsule of the present invention; and

FIGS. 3-5 are graphical representations of the dissolution performance of several formulations that may be used in embodiments.

### DETAILED DESCRIPTION OF THE DRAWINGS AND THE PRESENTLY PREFERRED EMBODIMENTS

The present invention is directed to methods and apparatus for monitoring pH levels in the gastrointestinal tract of a patient. The present invention is more particularly directed to methods and apparatuses for determining the duration of exposure of the esophagus to pH levels clinically significant for gastroesophageal reflux disease. The phrases “low pH,” “pH levels clinically significant for gastroesophageal reflux disease,” and the like refer to pH levels of about pH 4.

The terms “gastroesophageal device” or “gastroesophageal diagnostic device” and “gastrointestinal device” or “gastrointestinal diagnostic device” are all used to generally describe a device according to the present invention. The term “gastroesophageal” is used to specifically refer to the use of a device according to the present invention to monitor pH in the esophagus. The term “gastrointestinal” is used more generally to reflect that the scope of the present invention includes the use of a device according to the present invention to monitor pH in other areas of the gastrointestinal tract.

According to a preferred embodiment of the present invention, a system is described below that provides for a device comprising an ingestible but retrievable capsule for monitoring esophageal pH. The capsule contains a pH sensitive material distributed throughout and a cord is connected to the capsule. The cord has two ends, a proximal end and a distal end. For clarity, the end of the cord nearest to the capsule is referred to as the proximal end of the cord because when the capsule is inserted into the patient, this end is inserted first and is proximal to the LES. The distal end of the cord is the opposite end of the cord which remains accessible to the patient and/or the clinician. The amount of material in the capsule, its mass or volume, or both, is quantified before ingestion of the capsule by the patient. The patient retains the loose, or distal, end of the cord and swallows the capsule. The capsule is suspended in the lower esophagus and held in place with the cord. The capsule is preferably positioned in the lower one-third of the esophagus.

The position of the capsule can be monitored by fluoroscopic methods or by measurement of the cord itself, and can be controlled with the cord. Once properly positioned, the distal end of the cord may then be anchored in the patient's mouth or taped to the patient's face. The patient then proceeds with his normal daily activities. The material from the capsule is released over time as the capsule is exposed to pH levels clinically significant for acid reflux. After approximately twenty-four hours, the capsule is removed from the patient and the mass or volume of material, or both, is once again quantified. The decrease in the level of material in the capsule is correlated with the duration of time that the patient's esophagus is exposed to low pH. The longer the exposure to acidic conditions, the greater the decrease of material in the capsule. This information is used to diagnose gastroesophageal reflux.

An alternate way to determine the amount of the capsule not dissolved, i.e., that remained suspended in the patient, is to dissolve the capsule after retrieval and then use properties of the solution to determine the portion that remains after retrieval. For instance, the remaining portion may be ground up and placed into a known amount of solvent, such as alcohol or water. The color or turbidity of the solution may be compared to controls or known amounts. The amount that was placed into solution may then be calculated by an established technique, such as Beer's law. Beer's law relates the absorbance of light in a cell to the path length through the cell and the concentration of analyte in the cell. Alternatively, the change in conductivity of a solution of the amount remaining may be used.

The particular wavelength (color) of the light absorbed will differ with the chemistry and make-up of the capsule. The more of the capsule that remains and is dissolved, the more the light will be absorbed in the analytical technique used. For instance, an ultraviolet/visible/infrared (UV/VIS/IR) analyzer may be used. A turbidimeter, such as the Hach Model 18900 may be used to determine turbidity, the opacity of the solution, a measure of its ability to conduct light. As noted, the pH of the solution may also be used to determine the amount of material that remained at the end of the ingestion test. A conductivity meter, such as model CM-21P, from Analyticon Instruments Corp., Springfield, N.J., may be used to determine the electrical conductivity of the solution. The conductivity of the solution will also be a measure of the amount of capsule remaining at the end of the ingestion period.

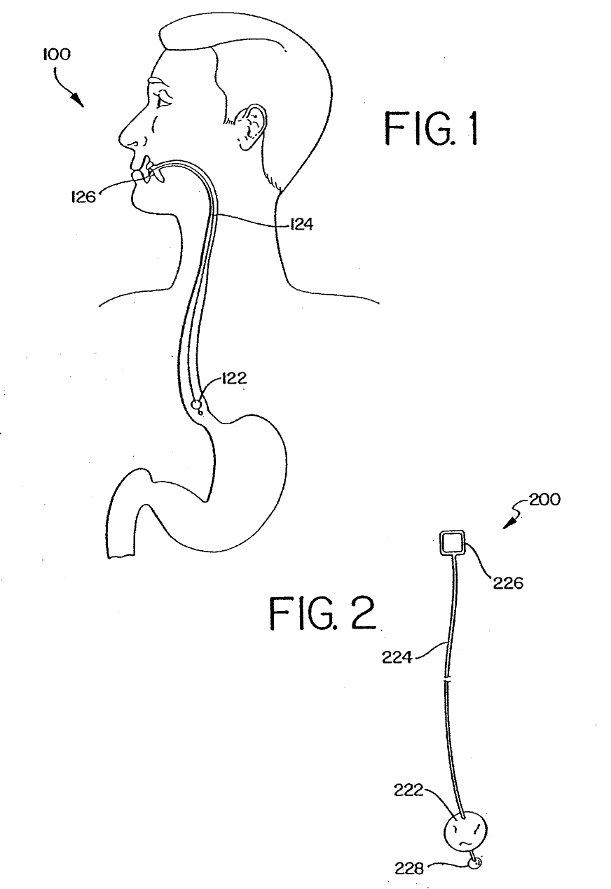

One preferred embodiment of a gastrointestinal diagnostic device 100 is shown in FIG. 1 which illustrates the use of the device 100 in a patient. The capsule 122 is suspended in the lower esophagus. A cord 124 is attached to the capsule. The cord 124 is used to suspend the capsule at the appropriate position in the esophagus of the patient and to retrieve the capsule. At the distal end of the cord, is an attachment member 126. The attachment member 126 is used to attach the cord to a location on the patient while the device is in use. The capsule or the cord may include a safe radiopaque material, such as barium sulfate, to ease x-ray or fluoroscopic detection of the position of the capsule. A small amount of radiopaque material may be added to the cord for x-ray or fluoroscopic detection, such as one or more strands of gold, platinum, or other radiopaque material.

Another embodiment of a gastroesophageal diagnostic device according to the present invention is shown in FIG. 2. The capsule 222 is sized and shaped to be readily swallowable. The capsule 222 is of a size that is easily ingestible and does not interfere with digestion or swallowing when positioned within a patient's esophagus. The capsule 222 is preferably made of a polymer, resin, or other non-toxic material which is biocompatible and inert. The capsule is preferably resistant to degradation at pH 4 and above, and subject to degradation when exposed to pH levels of about pH 4. The rate of degradation of the capsule at pH levels of about pH 4 is preferably slow enough so that a detectable amount of material is removed from the capsule, and a measurable amount of material also remains in the capsule, after exposure to clinically significant pH levels in the esophagus of a patient over a twenty-four hour period.

Many polymers have been developed for controlled-release delivery systems. Such systems rely on the chemical nature of a specific type of polymer to regulate the rate of polymer degradation under various conditions. Polymers commonly used to control release in a pH-dependent manner include derivatives of polyacrylates, derivatives of polymethacrylates, polyvinyl derivatives, and cellulose derivatives. For example, EUDRAGIT® polymers, manufactured by Rohm America, Inc., have been developed to be soluble in gastric juices but not at neutral pH levels. Hydrogels, or polymers that swell without dissolving, have also been developed for use in pH-dependent delivery. For additional information on pH-dependent biodegradable polymers, see Biomedical Polymers: Designed-to-Degrade Systems, S. W. Shalaby, Ed., Hanser, N.Y., 1994.

In one embodiment, a surface-eroding polymer composition is used. A surface-eroding polymer is a biodegradable polymer that degrades only at the exposed surface, so the release rate of mass and volume from the capsule is proportional to the surface area of the capsule. Examples of polymers that show surface erosion, as opposed to a bulk-erosion, include polyanhydrides and polyorthoesters. One alternative to controlling the rate of polymer degradation is to form a capsule and control its release by controlling polymer dissolution. The capsule can be surrounded by a pH-sensitive semipermeable membrane. The membrane would allow release of the content of the capsule only under conditions of pH levels of about pH 4.

In another embodiment, the capsule preferably is made from a uniform dispersion of pH-sensitive materials. The amount of material remaining in the capsule, by volume or mass, may be measured after exposure to the esophagus of a patient for about twenty-four hours or other selected period of time. After exposure, the amount of material dissolved, and the amount remaining, is dependent on the amount of time the capsule has been exposed to a clinically significant low pH.

In a preferred embodiment, the capsule is left in the esophagus of a patient for approximately twenty-four hours. A twenty-four hour period of time is preferred because it allows exposure of the capsule to the esophagus of a patient for a complete daily cycle. It is also possible, however, to leave the capsule positioned in a patient's esophagus for periods of time greater than twenty-four hours or less than twenty-four hours. In one alternate embodiment, the capsule is retained in the patient's esophagus for approximately twelve hours.

There are a number of materials that may be used to fabricate pH sensitive tablets or capsules. For instance, Rohm USA markets a series of Eudragit® materials, such as Eudragit® L and Eudragit® L100-55. These materials are typically poly methacrylate or polymethylmethacrylate materials that have been formulated for sensitivity to a particular pH, such as pH 6.0 or pH 5.5. When formulated with other known pharmaceutical excipients, the tablet may be tailored for dissolution at the desired pH without deleterious effects on the patient. Other useful excipients include polysaccharides, such as cellulose, methylcellulose, ethylcellulose, hydroxypropyl-cellulose, hydroxyethylcellulose, sodium carboxymethylcellulose (CMC), and so on. These materials are available from a number of suppliers, such as the Aqualon brand materials from Hercules, Inc., Wilmington, Del., USA.

Formulations have been developed for pH-sensitive release of drugs from pharmaceutical formulations. See, e.g., Daniel S. Kohane, et al., “pH-Triggered Release of Macromolecules from Spray-Dried Polymethacrylate Microparticles,” Pharm. Res., 20 (10): 1533-38 (2003); and Mingshi Yang et al., “A Novel pH-dependent gradient-release delivery system for nitrendipine, I. Manufacturing, evaluation in vitro and bioavailability in healthy dogs,” J. Contr. Rel. 98(2): 219-29 (2004). Formulations for a capsule for the detection of GERD may use pH-sensitive dissolution, at a selected pH, using combinations of lactic acid and cationic polymers based on dimethylaminoethyl methacrylate and neutral methacrylic esters. See, e.g., K. Malolepsza-Jarmolowska et al, “Studies on Gynaelogical Hydrophilic Lactic Acid Preparations,” Parmazie 58: 334-36 (2003). See also, Jinhe Li, et al., “In Vitro Evaluation of Dissolution Behavior for a Colon-Specific Drug Delivery System (CODES™) in Multi-pH Media Using United States Pharmacopeia Apparatus II and III,” AAPS PharmaSciTech 2002; 3(4) Article 33. Each of these articles is incorporated by reference as though the full text were reproduced in toto herein.

A preferred cationic polymer is Eudragit® E-100, from Rohm USA. Other cationic polymers may also be used, and other organic or carboxylic acids may be used, so long as the resulting combination dissolves at the desired pH which is useful for detecting and diagnosing GERD. Gastrosoluble cationic methacrylate copolymers, such as the Eudragit® E-12.5 and E PO copolymers may also be used. Capsules for ingestion are typically used for time-release of a medication. Such capsules typically include a protective coating, one or more excipients, and a binder to hold the capsule together.

The formulations may also include a lubricant, one or more plasticizers, an anti-sticking agent for the external surface, and a flavoring for better patient acceptance. The capsule preferably includes a lubricant such as, but not limited to, glyceryl monostearate, Myvaplex 600P, calcium stearate, or stearic acid. Lubricants are typically effective in an amount of from about 1 to 5 percent by weight of the coating, but more may be used. Plasticizers that may be used in embodiments include any of those known to those skilled in the art, including, but not limited to, acetyltributyl citrate, triacetin, acetylated monoglyceride, rape oil, olive oil, sesame oil, acetyltriethyl citrate, glycerin sorbitol, diethyloxalate, diethylmalate, diethylfumerate, dibutylsuccinate, diethylmalonate, dioctylphthalate, dibutylphthalate, dibutylsebacate, triethyl citrate, tributyl citrate, glyceroltributyrate, polyethylene glycol, propylene glycol and mixtures thereof. The plasticizer is typically present in an amount of from about 0.1% to about 3% by weight, but more may be used.

The tablet may be coated or covered with an anti-sticking agent, so that it may be more easily swallowed by a patient. Anti-sticking compounds include alkaline earth metal stearates, such as magnesium stearate or calcium stearate, or talc. Finally, a minute amount of flavoring, such as peppermint, may be used on the surface of the capsule, simply to make the capsule more acceptable to the patient. In the present application, a pH sensitive formulation is desired, with a binder for imparting strength to the capsule. Binders may include agents commonly known in the art such as polyvinyl pyrrolidone, hydroxyethyl cellulose, hydroxypropyl cellulose, low molecular weight hydroxypropyl methylcellulose (HPMC), polymethacrylate or ethyl cellulose. Such cellulosics are sold under the trade names of Methocel®, Aqualon®, Klucel®, Narosol®, among others.

Preferred are those cellulosics that have a relatively low threshold pH, and which can be combined with an organic acid, as described below. These include hydroxy-propyl methylcelluose phthalate having a threshold from about pH 4.5 to 4.8, and cellulose acetate trimellitate, having a threshold of about pH 4.8. Others that may be used include hydroxypropyl methylcelluose phthalate 5.0, threshold pH 5.0, and hydroxypropyl methylcelluose phthalate 55, with a threshold pH of 5.4. Others excipients may include cellulose acetate phthalate and polyvinyl acetate phthalate. Other cellulosics and other excipients, especially those with a lower pH threshold, may also be used. Most cellulosics are not soluble in water, but may be dissolved in organic solvents, such as anhydrous ethanol, as described below, or in other solvents.

In making capsules that are easily dissolvable at pH 4, a preferred embodiment is a 1:1 molar ratio of lactic acid and Eudragit® E-100. These ingredients are preferably present at about 7.5% total by weight of the capsule, but may range from 5 to 25%. The balance may be excipients as described above, such as methylcellulose. In order to minimize swelling, sorbitol may be added to the capsule, preferably in amounts from zero to twenty percent by weight.

A cord 224 is attached to or embedded in the capsule. The cord is long enough to extend distally outside the patient while the capsule is located at the desired position in the esophagus. The cord 224 may optionally include measuring indicia. The cord can be calibrated with a non-toxic dye to aid in the determination of the location of the capsule. As mentioned above, a small amount of radiopaque material may be added to the cord for x-ray or fluoroscopic detection, such as one or more strands of gold, platinum, or other radiopaque material. Like the material of the capsule 222, the cord 224 is preferably made of a material that is non-toxic and inert. The cord can be made of any thin, flexible material. Examples of materials that may be used for the cord 224 include string, nylon cord, fishing line, or various types of surgical sutures.

An attachment member 226 may be positioned at the distal end of the cord. Attachment 226 member allows anchoring of the distal end of the cord 224 and helps maintain the capsule 222 in its desired position in the patient. Attachment member 226 may be detachable from the cord or adjustable on the cord to allow the attachment member 226 to be easily positioned at the desired location. Attachment member 226 is preferably a material that is non-toxic and inert. The exact shape of attachment member 226 depends on the specific attachment site. The attachment site is preferably at a location that is relatively easy for the clinician to access and that does not significantly interfere with the normal functioning or the daily routine of the patient.

A manufacturing process is used to produce tablets with the desired pH-sensitive dissolution properties. In a preferred embodiment, the required amount of Eudragit® E-100 is poured over a weighed amount of lactic acid. The mass is stirred to obtain a homogenous suspension. The mixture is then left overnight for about 24 hours, until a clear, thick liquid forms. This liquid is then combined with methylcellulose. The mixture is mixed to obtain a homogenous mass and dried at about 40° C. to evaporate water from the lactic acid. The result is a dry mass. Tablets or capsules are then formed directly. An attachment member may be molded into the tablet so that a cord may be attached to retrieve the capsule.

An alternative process also incorporates glycerol, which enables the swallowed tablets to swell quickly and produce a gel. These tablets are even more sensitive to pH than tablets without glycerol. To produce tablets with glycerol, a somewhat modified process is followed. Glycerol is dissolved in 96% ethanol, at a ratio of about 50 ml of ethanol for 11 g of methylcellulose in an anhydrous environment. The homogenous mixture produced by thus wetting the methylcellulose with ethanol is dried at 40° C. and then passed through a 0.5 mm sieve. The process as described above is then continued, beginning with a step of mixing the dried methylcellulose/ethanol/glycerol mixture with Eudragit® E-100/lactic acid mixture, and proceeding with the steps through tabletting. Table 1, below, from one of the references mentioned above, discloses the pH performance of several combinations of the above-mentioned materials, which dissolve from about pH 3.8 to about pH 4.4, using molar ratios of lactic acid to Eudragit® E-100 from 1:1 to 3:1. Many other materials and combinations are possible.

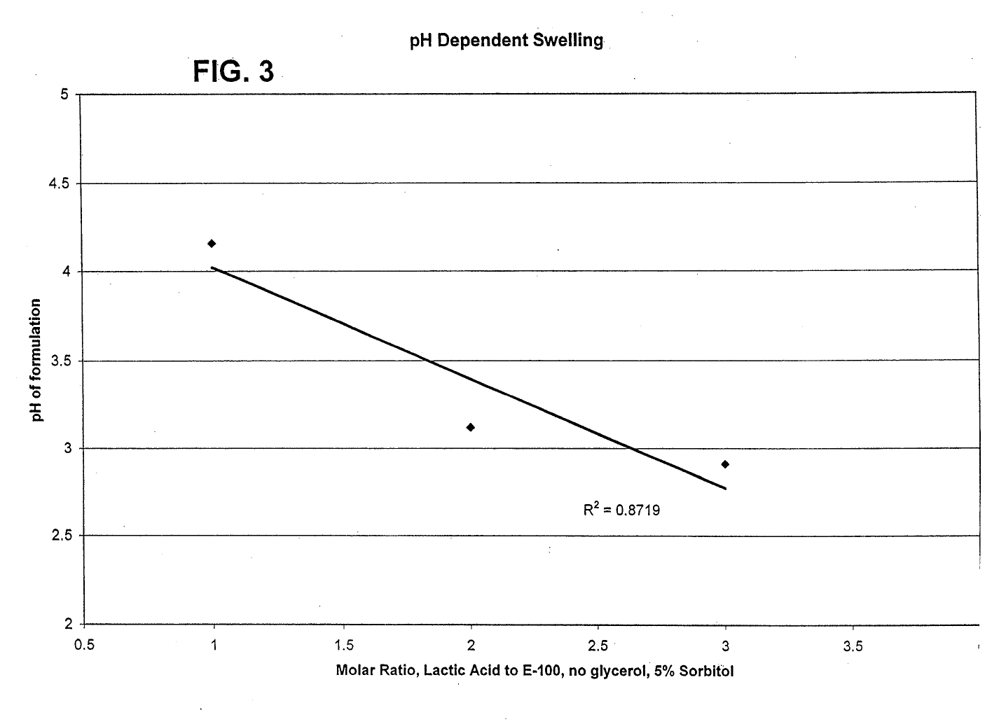

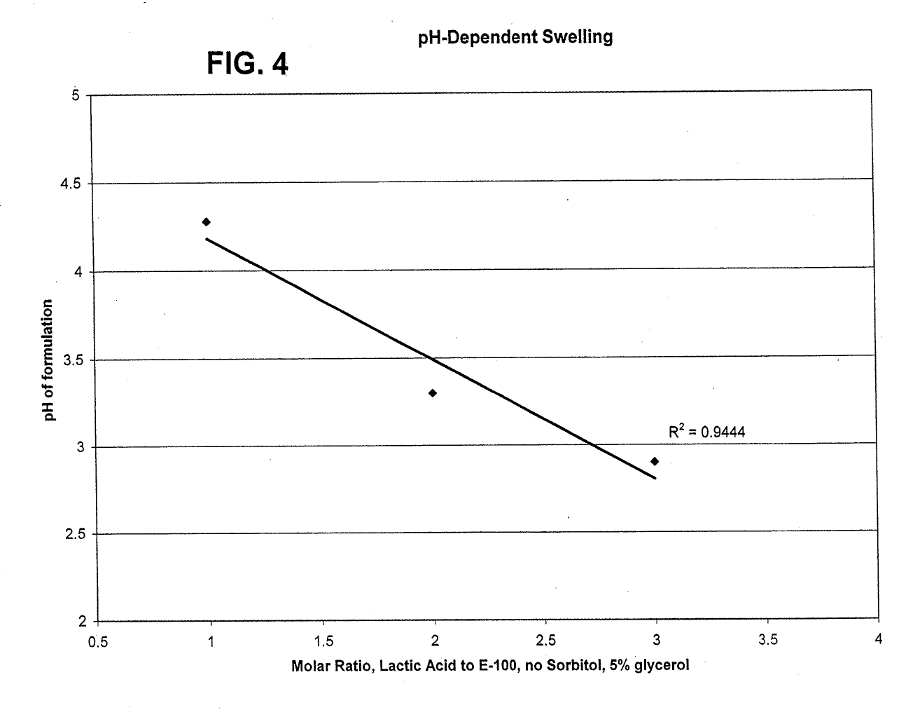

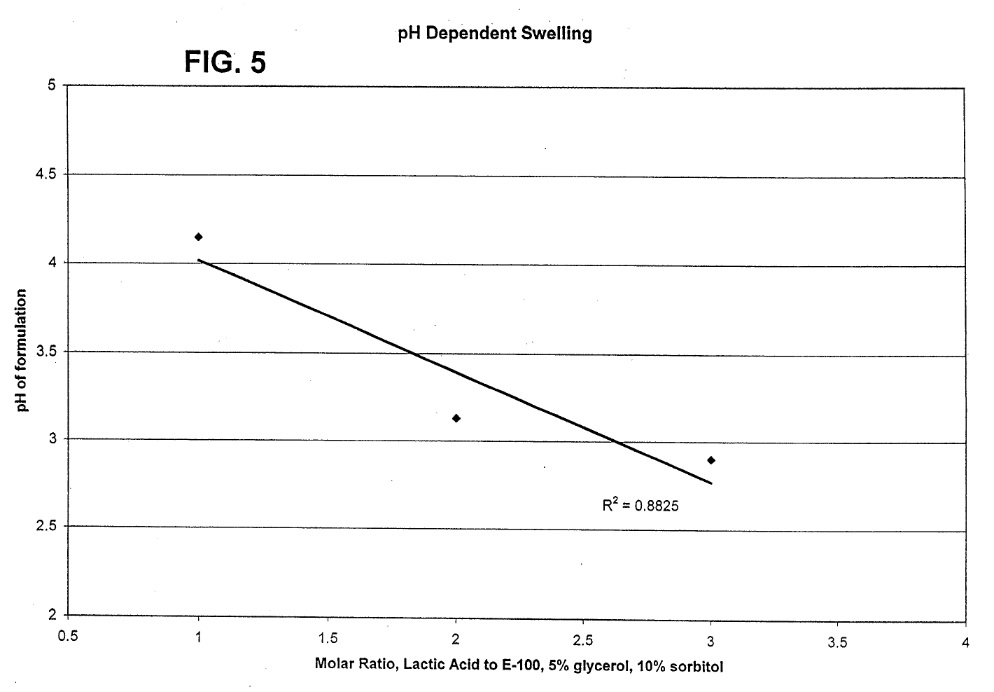

The pH of the resulting tablets, and their resulting expected dissolution performance is depicted graphically in FIGS. 3-5. The correlation, R², between the pH and the molar ratio of the lactic acid (pKa 3.86) is approximately 0.9, with a range from 0.87 to 0.94, for several levels of glycerol and sorbitol. This is a very high correlation, and is relatively independent of both glycerol and sorbitol content, suggesting that it is the molar ratio alone that determines pH performance.

Accordingly, the pH performance of the resulting tablets may be adjusted to a pH just below pH 4 by selected a molar ratio of just above unity, say about 1.1, and just below 1.25, about 1.2. Other acids may also be used, with appropriate adjustment of the ratio of acid to E-100 or other acrylate or methacrylate polymer. Then pKa of the acid may be used as a general guide, with acids with a high pKa (a weaker acid) expected to require a slightly higher ratio, than that of lactic acid. For example, citric acid has a first pKa of 3.06, less than that of lactic acid (3.86), indicating that it is a stronger acid that lactic acid. This suggests that formulations using this acid should use an acid/methacrylate ratio lower than that used for lactic acid. On the other hand, acids with high pKa's, such as ascorbic acid (first pKa 4.10), or benzoic acid (pKa 4.20), should probably used an acid/methacrylate ratio slightly higher than that used for lactic acid. Of course, the ratios must also be adjusted appropriately if the acid yields more than one proton.

TABLE 1
Influence of the composition of the tablet on pH, dynamic
viscosity and swelling properties of tablets

| | MC | GL | SR | LA | E | | | V | HG |
|---|---|---|---|---|---|---|---|---|---|
| BN | (g) | (g) | (g) | (g) | (g) | LA:E | pH | (mPas) | (mm) |
| Ia | 87.35 | 5.0 | 0.0 | 2.24 | 5.41 | 1:1 | 4.28 | 148.7 | 25.0 |
| Ib | 85.11 | 5.0 | 0.0 | 4.48 | 5.41 | 2:1 | 3.30 | 130.3 | 22.5 |
| Ic | 82.87 | 5.0 | 0.0 | 6.72 | 5.41 | 3:1 | 2.90 | 108.4 | 25.0 |
| IIa | 82.35 | 5.0 | 5.0 | 2.24 | 5.41 | 1:1 | 4.09 | 86.6 | 18.5 |
| IIb | 80.11 | 5.0 | 5.0 | 4.48 | 5.41 | 2:1 | 3.11 | 78.7 | 20.5 |
| IIc | 77.87 | 5.0 | 5.0 | 6.72 | 5.41 | 3:1 | 2.82 | 72.6 | 21.0 |
| IIIa | 77.35 | 5.0 | 10.0 | 2.24 | 5.41 | 1:1 | 4.15 | 71.7 | 19.5 |
| IIIb | 75.11 | 5.0 | 10.0 | 4.48 | 5.41 | 2:1 | 3.13 | 64.7 | 21.0 |
| IIIc | 72.87 | 5.0 | 10.0 | 6.72 | 5.41 | 3:1 | 2.90 | 54.2 | 18.0 |
| IVa | 72.35 | 5.0 | 15.0 | 2.24 | 5.41 | 1:1 | 4.18 | 71.7 | 19.0 |
| IVb | 70.11 | 5.0 | 15.0 | 4.48 | 5.41 | 2:1 | 3.15 | 64.7 | 20.0 |
| IVc | 67.87 | 5.0 | 15.0 | 6.72 | 5.41 | 3:1 | 2.97 | 54.2 | 19.0 |
| Va | 82.35 | 10.0 | 0.0 | 2.24 | 5.41 | 1:1 | 4.20 | 136.4 | 21.0 |
| Vb | 80.11 | 10.0 | 0.0 | 4.48 | 5.41 | 2:1 | 3.14 | 121.6 | 21.5 |
| Vc | 77.87 | 10.0 | 0.0 | 6.72 | 5.41 | 3:1 | 2.88 | 96.2 | 24.5 |
| VIa | 87.35 | 0.0 | 5.0 | 2.24 | 5.41 | 1:1 | 4.16 | 106.7 | 20.0 |
| VIb | 85.11 | 0.0 | 5.0 | 4.48 | 5.41 | 2:1 | 3.12 | 101.4 | 18.0 |
| VIc | 82.87 | 0.0 | 5.0 | 6.72 | 5.41 | 3:1 | 2.91 | 96.2 | 13.5 |
| VIIa | 82.35 | 0.0 | 10.0 | 2.24 | 5.41 | 1:1 | 4.21 | 90.9 | 16.5 |
| VIIb | 80.11 | 0.0 | 10.0 | 4.48 | 5.41 | 2:1 | 3.20 | 87.4 | 17.5 |
| VIIc | 77.87 | 0.0 | 10.0 | 6.72 | 5.41 | 3:1 | 2.94 | 83.9 | 17.0 |
| VIIIa | 77.35 | 0.0 | 15.0 | 2.24 | 5.41 | 1:1 | 4.27 | 84.4 | 17.0 |
| VIIIb | 75.11 | 0.0 | 15.0 | 4.48 | 5.41 | 2:1 | 3.26 | 70.0 | 15.0 |
| VIIIc | 72.87 | 0.0 | 15.0 | 6.72 | 5.41 | 3:1 | 2.98 | 59.5 | 14.0 |
| IXa | 72.35 | 0.0 | 20.0 | 2.24 | 5.41 | 1:1 | 4.29 | 68.2 | 15.0 |
| IXb | 70.11 | 0.0 | 20.0 | 4.48 | 5.41 | 2:1 | 3.29 | 53.3 | 14.0 |
| IXc | 67.87 | 0.0 | 20.0 | 6.72 | 5.41 | 3:1 | 2.93 | 45.5 | 11.0 |

BN—Batch Number, MC—Methylcellulose, GL—Glycerol, SR—Sorbitol, LA—Lactic Acid, E—Eudragit* E-100, LA:E—Molar Ratio of Lactic Acid to Eudragit, V—Dynamic viscosity, pH is the pH of the resulting gel; HG—Height of gel column after 10 min. of measurement. Excerpted from Studies on Gynaecological Hydrophilic Lactic Acid Preparations.

In one embodiment, as seen in FIG. 1, the attachment site is at one or more of the patient's teeth. In this case, the attachment member 126 (226 in FIG. 2) may be a cap that fits over one or more of the patient's teeth. The cap can be removably affixed or cemented to one or more of the patient's teeth to increase its stability. Alternatively, the attachment member 226 may simply be part of the cord 224 itself, particularly if the attachment site is one or more of the patient's teeth. For example, the attachment member 226 may be a loop in the cord, positioned at the distal end of the cord, which wraps around one or more of the patient's teeth and holds the device 200 in place. Alternatively, the cord can be taped to the patient's face or to some other area.

There may optionally be a weight 228 positioned adjacent to the capsule 222 which helps guide the capsule 222 down the gastrointestinal tract when the capsule 222 is ingested. The weight 228 also helps maintain the capsule 222 in its desired position while in place in the patient. The weight 228 is preferably made of an inert, nontoxic, material with a density greater than that of the capsule. The weight is preferably positioned at the proximal end of the cord, but may also be positioned distal to the capsule.

According to a preferred embodiment, the gastroesophageal diagnostic device is utilized in the following manner. The capsule 222 is first measured via its mass, or volume, or both. The individual capsule may, for example, be weighed on an accurate electronic scale to determine its mass. The volume of the capsule may also be determined by any of several known displacement techniques. The patient then retains the distal end of the cord 224 near the attachment member 226 and ingests the capsule 222. The capsule 222 is preferably positioned in the lower one-third of the esophagus, approximately five centimeters distal to the lower esophageal sphincter (LES). The position of the capsule 222 can be determined in several ways. First, if the cord 224 is calibrated with a non-toxic dye, the approximate position of the capsule 222 may be determined by making an estimate based on the length of cord 224 which has been ingested. Alternatively, a radiopaque marker on the capsule, or a radiopaque strand in the cord, can be detected with x-rays, or fluoroscopy.

Once the capsule 222 is located at the appropriate position, the attachment member 226 is positioned and fixed in place. For example, if the attachment member 226 is a cap that fits over a tooth of the patient, the clinician may cement the cap over the tooth to hold the cap in place while the patient is undergoing the diagnostic procedure. The attachment member 226 is preferably detachable from the cord or adjustable on the cord to allow the attachment member 226 to be positioned easily. The length of cord 224 necessary to properly position the capsule 222 depends on the size and shape of the patient. Once the proper positioning of the capsule 222 is determined, the adjustable attachment member 226 may be positioned for anchoring the device 200 in place.

At this point, the patient is free to engage in his normal daily activities. Preferably, the gastroesophageal diagnostic device 200 remains in place in the patient for approximately twenty-four hours. Allowing the patient to go through an entire twenty-four hour cycle, including eating, sleeping and other daily activities, provides a more accurate determination of the amount of time that the esophagus is exposed to low pH. Since the device is small, comfortable, and easy to use, there is little to no interference with the patient's normal routine.

After twenty-four hours, the device 200 is removed from the patient. The attachment member 226 is disassociated from the point of attachment, and the cord 224 is used to pull the capsule 222 up from the patient's esophagus. Then the capsule is measured to determine its mass or volume, or both, and the change in the mass or volume is used to determine the duration of time, over the twenty-four hour diagnostic period, that the esophagus is exposed to low pH conditions caused by reflux of stomach acid into the esophagus.

To determine the total time of exposure of the esophagus to low pH, the decrease in mass or volume of the capsule is compared to a series of standards. The standards are generated by exposing capsules 222 to samples with the chemical characteristics of gastric acid, including a pH of about pH 4. A standard curve is generated by measuring the mass or volume remaining in a series of capsules after various times of exposure to pH levels of about pH 4. The loss of mass or volume that takes place in the capsule over a twenty-four hour period with no exposure to low pH is also taken into account when generating a standard curve. Standards may be generated using either the decrease in mass in the capsule or the decrease of volume of the capsule after exposure. By comparing either the decrease in volume in the capsule or the decrease in mass of the capsule to the corresponding set of standards, a determination can be made of the duration of time that the capsule was exposed to pH levels of about pH 4, which are clinically significant for gastroesophageal reflux disease. This information may then be used in making a diagnosis regarding gastroesophageal reflux disease.

As noted above, the preferred formulation for the capsule includes lactic acid and the Eudragit E-100 co-polymer. Other acids may also be used to make pH-sensitive formulations. The acids must of course be medically acceptable for ingestion by a patient, and must not be harmful to the patient. Acids with a pKa in the range of about 3 to about 5 are preferred, such as salicylic acid, hemimellitic acid, 1,4-piperazinebis-(ethanesulfonic) acid, tartaric acid, fumaric acid, glycylglycine, citric acid, cyclopentanetetra-1,2,3,4-carboxylic acid, pyromellitic acid, trimesic acid, mellitic acid, dimethylmalonic acid, mandelic acid, butane-1,2,3,4,-tetracarboxylic acid, ascorbic acid, benzoic acid, acetic acid, and propionic acid. Other acids may be used, combined with a methacrylate polymer or co-polymer to yield a tablet or capsule that dissolves in the desired pH range, below about pH 4, or from about pH 3.7 to about pH 4.0. In addition, combinations of acids or combinations of copolymers may be used instead, so long as the resultant capsule is sensitive to, i.e., dissolves in, the desired pH range.

In addition to the formulations mentioned above, many other formulations are possible. The formulations described above first required a reaction between the acid and the methylcellulose, before the addition of the lactic acid and the other components. It is not clear that the methacrylate polymers are required in order for the formulation to perform properly at reduced pH. Accordingly, additional embodiments may be formulated without the Eudragit® polymers, using a cellulose, such as sodium carboxymethyl cellulose, or a polysaccharide, such as sodium alginate.

Finally, an ingestible, non-harmful dye or pigment may be included with the capsule to aid in the analysis after the test period. Many such dyes are available, including those approved by the Food and Drug Administration (FDA) and typically named as a particular FD&C (Food Drug & Cosmetic) approved number. Acidic varieties are preferred, as well as those from which no adverse reactions are expected. Preferred may be FD&C Red #3 (erythrosine B) and methylene blue. Many other dyes may be used instead.

While the invention has been described and illustrated with reference to specific illustrative embodiments thereof, it is not intended that the invention be limited to those illustrative embodiments. Those skilled in the art will recognize that variations and modifications can be made without departing from the spirit of the invention. It is therefore intended to include within the invention all such variations and modifications that fall within the scope of the appended claims and equivalents thereof.

It is therefore intended that the foregoing detailed description be regarded as illustrative rather than limiting, and that it be understood that it is the following claims, including all equivalents, that are intended to define the spirit and scope of this invention.

1. A device for determining a duration of exposure of a patient's esophagus to pH levels clinically significant for gastroesophageal reflux disease comprising:
a capsule subject to pH-dependent degradation at pH of about pH 4, and being of a size to be readily swallowable, wherein the capsule comprises a reaction product of a cellulose, a polysaccharide, or a cationic polymer with an organic acid; and
a cord, the cord having a proximal end and a distal end, the proximal end being connected to the capsule, whereby the cord allows positioning and retrieval of the capsule.

2. A device according to claim 1 further comprising an attachment member, the attachment member being connected to the distal end of the cord, whereby the attachment member allows the distal end of the cord to be fixed in a selected position.

3. A device according to claim 1 further comprising a weight, the weight being positioned on the cord and adjacent to the capsule.

4. A device according to claim 1 wherein the cord includes measuring indicia along the length of the cord, whereby the measuring indicia allow measurement of the length of cord inserted into a patient.

5. A device according to claim 1 wherein the capsule further comprises a small amount of a pigment or dye.

6. A device according to claim 1 wherein at least one of the capsule and the cord further comprises a radiopaque material.

7. The device according to claim 1 wherein the capsule includes a reaction product of lactic acid and a cationic polymer based on dimethylaminoethyl methacrylate and neutral methacrylic esters.

8. The device according to claim 1 wherein the capsule includes a reaction product of lactic acid and a cationic polymer based on dimethylaminoethyl methacrylate and neutral methacrylic esters, said acid and polymer present in a molar ratio from about 1:1 to about 1:1.25.

9. The device according to claim 1 wherein the capsule includes a reaction product of a polymer and an organic acid with a pKa from about 3 to about 5.

10. The device according to claim 1 wherein the capsule includes a reaction product of a polymer and an acid selected from the group consisting of salicylic acid, hemimellitic acid, 1,4-piperazinebis-(ethanesulfonic) acid, tartaric acid, fumaric acid, glycylglycine, citric acid, cyclopentanetetra-1,2,3,4-carboxylic acid, pyromellitic acid, ascorbic acid, trimesic acid, mellitic acid, dimethylmalonic acid, mandelic acid, benzoic acid, acetic acid and propionic acid.

11. A device for determining a duration of exposure of a patient's esophagus to pH levels clinically significant for gastroesophageal reflux disease comprising:
a capsule subject to pH-dependent degradation at pH of about pH 4, and of a size to be readily swallowable, the capsule further comprising a reaction product of an acid and a cationic polymer based on dimethylaminoethyl methacrylate and neutral methacrylic esters; and
a cord, the cord having a proximal end and a distal end, the proximal end being connected to the capsule, whereby the cord allows positioning and retrieval of the capsule.

12. The device of claim 11, wherein a molar ratio of the acid and the cationic polymer is about 1.1 to about 1.2.

13. The device of claim 11, wherein the acid is an organic acid having a pKa from about 3 to about 5.

14. The device of claim 11, wherein the acid and the cationic polymer are present at a total of about 5-15 percent by weight.

15. The device of claim 11, wherein the capsule or the cord further comprises a radiopaque marker and optionally, a small amount of a pigment or dye.

16. A method of detecting gastroesophageal reflux disease, the method comprising:
providing a gastroesophageal diagnostic device, said gastroesophageal diagnostic device comprising a capsule having a cord attached thereto, the capsule being subject to pH-dependent degradation at pH levels of about pH 4;
measuring at least one of a mass and a volume of the capsule;
introducing the gastroesophageal diagnostic device into an esophagus of a patient;
positioning the gastroesophageal diagnostic device in the esophagus of the patient such that the capsule is positioned in the lower one-third of the esophagus;
leaving the gastroesophageal diagnostic device in the esophagus of the patient for a time, whereby the capsule loses a portion of at least one of the mass and the volume in a pH-dependent manner;
removing the gastroesophageal diagnostic device from the patient after the time; and
determining an exposure time during which the capsule was exposed to pH levels of about pH 4, wherein a reduction in the amount of the mass or the volume correlates to the exposure time during which the capsule was exposed to pH levels of about pH 4, and wherein exposure of the capsule to pH levels of about pH 4 for at least a determined percent of the time the device was left in the esophagus is indicative of gastroesophageal reflux disease.

17. The method of claim 16 wherein the determined percent of the time is at least about five percent.

18. The method of claim 16 wherein the exposure time is determined by measuring the mass or the volume of the capsule remaining after the time.

19. The method of claim 16 wherein the exposure time is determined by a technique selected from the group consisting of pH, color, conductivity or turbidity.

20. The method of claim 16 wherein the time for leaving the gastroesophageal diagnostic device in the esophagus of the patient is about 24 hours.

21. A method of detecting gastroesophageal reflux disease, the method comprising:
providing a gastroesophageal diagnostic device, said gastroesophageal diagnostic device comprising a capsule having a cord attached thereto, the capsule being subject to pH-dependent degradation at pH levels of about pH 4;
introducing the gastroesophageal diagnostic device into an esophagus of a patient;
positioning the gastroesophageal diagnostic device in the esophagus of the patient such that the capsule is positioned in the lower one-third of the esophagus;
leaving the gastroesophageal diagnostic device in the esophagus of the patient for a time, whereby the capsule loses a portion of at least one of a mass and a volume of the device in a pH-dependent manner;
removing the gastroesophageal diagnostic device from the patient after the time; and
determining an exposure time during which the capsule was exposed to pH levels of about pH 4 by a method selected from the group consisting of color, turbidity, pH, and conductivity, wherein exposure of the capsule to pH levels of about pH 4 for a minimum time during the time the device is left in the esophagus is indicative of gastroesophageal reflux disease.
